(19) United States
(12) Patent Application Publication
(10) Pub. No.: US 2020/0352658 A1
(43) Pub. Date: Nov. 12, 2020

(54) USER INTERFACE FOR ROBOTIC SURGICAL SYSTEM

(71) Applicant: Medrobotics Corporation, Raynham, MA (US)

(72) Inventors: Joseph M. Johnson, Norwood, MA (US); James Wilson, Norwood, MA (US); Alexander Patt Broerman, Somerville, MA (US); Duncan Freake, Somerville, MA (US); Ashley Whitney, Somerville, MA (US); Stuart D. Perry, Wellesley Hills, MA (US); Guarav Rohatgi, Waltham, MA (US)

(21) Appl. No.: 16/960,158

(22) PCT Filed: Jan. 7, 2019
(86) PCT No.: PCT/US19/12478

§ 371 Date: Jul. 6, 2020

Related U.S. Application Data
(60) Provisional application No. 62/614,223, filed on Jan. 5, 2018.
(60) Provisional application No. 62/614,224, filed on Jan. 5, 2018.
(60) Provisional application No. 62/614,240, filed on Jan. 5, 2018.

(51) Int. Cl. A61B 34/00; A61B 34/30; A61B 90/50

(52) U.S. Cl. CPC A61B 34/25; A61B 34/30; A61B 2034/301; A61B 34/76; A61B 90/50; A61B 34/74

(57) ABSTRACT

A system for performing a medical procedure on a patient includes an articulating probe assembly and at least one tool. The articulating probe assembly comprises an inner probe comprising multiple articulating inner links, an outer probe surrounding the inner probe and comprising multiple articulating outer links, and at least two working channels that exit a distal portion of the probe assembly. The at least one tool is configured to translate through one of the at least two working channels. A user interface controls the articulating probe assembly.

### RELATED APPLICATIONS

This application claims the benefit of U.S. Provisional Application No. 62/613,899, filed Jan. 5, 2018, the content of which is incorporated herein by reference in its entirety.

This application claims the benefit of U.S. Provisional Application No. 62/614,223, filed Jan. 5, 2018, the content of which is incorporated herein by reference in its entirety.

This application claims the benefit of U.S. Provisional Application No. 62/614,224, filed Jan. 5, 2018, the content of which is incorporated herein by reference in its entirety.

This application claims the benefit of U.S. Provisional Application No. 62/614,228, filed Jan. 5, 2018, the content of which is incorporated herein by reference in its entirety.

This application claims the benefit of U.S. Provisional Application No. 62/614,225, filed Jan. 5, 2018, the content of which is incorporated herein by reference in its entirety.

This application claims the benefit of U.S. Provisional Application No. 62/614,240, filed Jan. 5, 2018, the content of which is incorporated herein by reference in its entirety.

This application claims the benefit of U.S. Provisional Application No. 62/614,235, filed Jan. 5, 2018, the content of which is incorporated herein by reference in its entirety.

This application is related to U.S. Provisional Application No. 61/921,858, filed Dec. 30, 2013, the content of which is incorporated herein by reference in its entirety.

This application is related to PCT Application No PCT/US2014/071400, filed Dec. 19, 2014, PCT Publication No. WO2015/102939, the content of which is incorporated herein by reference in its entirety.

This application is related to U.S. patent application Ser. No. 14/892,750, filed Nov. 20, 2015, U.S. Publication No. 2016/0256226, now U.S. Pat. No. 10,004,568 issued on Jun. 26, 2018, the content of which is incorporated herein by reference in its entirety.

This application is related to U.S. patent application Ser. No. 15/899,826, filed Feb. 20, 2018, U.S. Publication No. 2018/0250095 the content of which is incorporated herein by reference in its entirety.

This application is related to U.S. Provisional Application No. 61/406,032, filed Oct. 22, 2010, the content of which is incorporated herein by reference in its entirety.

This application is related to PCT Application No PCT/US2011/057282, filed Oct. 21, 2011, PCT Publication No. WO2012/054829, the content of which is incorporated herein by reference in its entirety.

This application is related to U.S. patent application Ser. No. 13/880,525, filed Apr. 19, 2013, U.S. Publication No. 2014/0005683, now U.S. Pat. No. 8,992,421, issued on Mar. 31, 2015, the content of which is incorporated herein by reference in its entirety.

This application is related to U.S. patent application Ser. No. 14/587,166, filed Dec. 31, 2014, U.S. Publication No. 2015/0313449, the content of which is incorporated herein by reference in its entirety.

This application is related to U.S. Provisional Application No. 61/492,578, filed Jun. 2, 2011, the content of which is incorporated herein by reference in its entirety.

This application is related to PCT Application No. PCT/US2012/040414, filed Jun. 1, 2012, PCT Publication No. WO2012/167043, the content of which is incorporated herein by reference in its entirety.

This application is related to U.S. patent application Ser. No. 14/119,316, filed Nov. 21, 2013, U.S. Publication No. 2014/0094825, the content of which is incorporated herein by reference in its entirety.

This application is related to U.S. Provisional Application No. 62/504,175, filed May 10, 2017, the content of which is incorporated herein by reference in its entirety.

This application is related to PCT Application No. PCT/US2018/031774, filed May 9, 2018, PCT Publication No. WO2018/0020898, the content of which is incorporated herein by reference in its entirety.

This application is related to U.S. Provisional Application No. 61/412,733, filed Nov. 11, 2010, the content of which is incorporated herein by reference in its entirety.

This application is related to PCT Application No PCT/US2011/060214, filed Nov. 10, 2011, PCT Publication No. WO2012/078309, the content of which is incorporated herein by reference in its entirety.

This application is related to U.S. patent application Ser. No. 13/884,407, filed May 9, 2013, U.S. Publication No. 2014/0012288, now U.S. Pat. No. 9,649,163, issued on May 16, 2017, the content of which is incorporated herein by reference in its entirety.

This application is related to U.S. patent application Ser. No. 15/587,832, filed May 5, 2017, U.S. Publication No. 2018/0021095, the content of which is incorporated herein by reference in its entirety.

This application is related to U.S. Provisional Application No. 61/472,344, filed Apr. 6, 2011, the content of which is incorporated herein by reference in its entirety.

This application is related to PCT Application No. PCT/US2012/032279, filed Apr. 5, 2012, PCT Publication No. WO2012/138834, the content of which is incorporated herein by reference in its entirety.

This application is related to U.S. patent application Ser. No. 14/008,775, filed Sep. 30, 2013, U.S. Publication No. 2014/0046305, now U.S. Pat. No. 9,962,179, issued on May 8, 2018, the content of which is incorporated herein by reference in its entirety.

This application is related to U.S. patent application Ser. No. 14/944,665, filed Nov. 18, 2015, U.S. Publication No.: 2016/0066938, the content of which is incorporated herein by reference in its entirety.

This application is related to U.S. patent application Ser. No. 14/945,685, filed Nov. 19, 2015, U.S. Publication No. 2016/0066939, the content of which is incorporated herein by reference in its entirety.

This application is related to U.S. Provisional Application No. 61/534,032 filed Sep. 13, 2011, the content of which is incorporated herein by reference in its entirety.

This application is related to PCT Application No. PCT/US2012/054802, filed Sep. 12, 2012, PCT Publication No. WO2013/039999, the content of which is incorporated herein by reference in its entirety.

This application is related to U.S. patent application Ser. No. 14/343,915, filed Mar. 10, 2014, U.S. Publication No. 2014/0371764, now U.S. Pat. No. 9,757,856, issued on Sep. 12, 2017, the content of which is incorporated herein by reference in its entirety.

This application is related to U.S. patent application Ser. No. 15/064,043, filed Mar. 8, 2016, U.S. Publication No. 2016/0262840, now U.S. Pat. No. 9,572,628, issued on Feb. 21, 2017, the content of which is incorporated herein by reference in its entirety.

This application is related to U.S. patent application Ser. No. 15/684,268, filed Aug. 23, 2017, U.S. Publication No. 2017/0368681, the content of which is incorporated herein by reference in its entirety.

This application is related to U.S. Provisional Application No. 61/368,257, filed Jul. 28, 2010, the content of which is incorporated herein by reference in its entirety.

This application is related to PCT Application No PCT/US2011/044811, filed Jul. 21, 2011, PCT Publication No. WO2012/015659, the content of which is incorporated herein by reference in its entirety.

This application is related to U.S. patent application Ser. No. 13/812,324, filed Jan. 25, 2013, U.S. Publication No. 2014/0012287, now U.S. Pat. No. 9,901,410, issued on Feb. 27, 2018, the content of which is incorporated herein by reference in its entirety.

This application is related to U.S. patent application Ser. No. 15/874,189, filed Jan. 18, 2018, U.S. Publication No. 2018-0206923 the content of which is incorporated herein by reference in its entirety.

This application is related to U.S. Provisional Application No. 61/578,582, filed Dec. 21, 2011, the content of which is incorporated herein by reference in its entirety.

This application is related to PCT Application No. PCT/US2012/070924, filed Dec. 20, 2012, PCT Publication No. WO2013/096610, the content of which is incorporated herein by reference in its entirety.

This application is related to U.S. patent application Ser. No. 14/364,195, filed Jun. 10, 2014, U.S. Publication No. 2014/0318299, now U.S. Pat. No. 9,364,955 issued on Jun. 14, 2016, the content of which is incorporated herein by reference in its entirety.

This application is related to U.S. patent application Ser. No. 15/180,503, filed Jun. 13, 2016, U.S. Publication No. 2017/0015007, now U.S. Pat. No. 9,821,477, issued on Nov. 21, 2017, the content of which is incorporated herein by reference in its entirety.

This application is related to U.S. patent application Ser. No. 15/786,901, filed Oct. 18, 2017, U.S. Publication No. 2018/0161992, the content of which is incorporated herein by reference in its entirety.

This application is related to U.S. Provisional Application No. 61/681,340, filed Aug. 9, 2012, the content of which is incorporated herein by reference in its entirety.

This application is related to PCT Application No. PCT/US2013/054326, filed Aug. 9, 2013, PCT Publication No. WO2014/026104, the content of which is incorporated herein by reference in its entirety.

This application is related to U.S. patent application Ser. No. 14/418,993, filed Feb. 2, 2015, U.S. Publication No. 2015/0282835, now U.S. Pat. No. 9,675,380 issued on Jun. 13, 2017, the content of which is incorporated herein by reference in its entirety.

This application is related to U.S. patent application Ser. No. 15/619,875, filed Jun. 12, 2017, U.S. Publication No. 2018/0021060, the content of which is incorporated herein by reference in its entirety.

This application is related to U.S. Provisional Application No. 61/751,498, filed Jan. 11, 2013, the content of which is incorporated herein by reference in its entirety.

This application is related to PCT Application No. PCT/US2014/010808, filed Jan. 9, 2014, PCT Publication No. WO2014/110218, the content of which is incorporated herein by reference in its entirety.

This application is related to U.S. patent application Ser. No. 14/759,020, filed Jul. 2, 2015, U.S. Publication No. 2015/0342690, the content of which is incorporated herein by reference in its entirety.

This application is related to U.S. Provisional Application No. 61/656,600, filed Jun. 7, 2012, the content of which is incorporated herein by reference in its entirety.

This application is related to PCT Application No. PCT/US2013/043858, filed Jun. 3, 2013, PCT Publication No. WO2013/184560, the content of which is incorporated herein by reference in its entirety.

This application is related to U.S. patent application Ser. No. 14/402,224, filed Nov. 19, 2014, U.S. Publication No. 2015/0150633, the content of which is incorporated herein by reference in its entirety.

This application is related to U.S. Provisional Application No. 61/825,297, filed May 20, 2013, the content of which is incorporated herein by reference in its entirety.

This application is related to PCT Application No. PCT/US2013/038701, filed May 20, 2014, PCT Publication No. WO2014/189876, the content of which is incorporated herein by reference in its entirety.

This application is related to U.S. patent application Ser. No. 14/888,541, filed Nov. 2, 2015, U.S. Publication No. 2016/0074028, now U.S. Pat. No. 9,517,059, issued on Dec. 13, 2016, the content of which is incorporated herein by reference in its entirety.

This application is related to U.S. patent application Ser. No. 15/350,549, filed Nov. 14, 2016, U.S. Publication No. 2017/0119364, now U.S. Pat. No. 10,016,187, issued on Jul. 10, 2018, the content of which is incorporated herein by reference in its entirety.

This application is related to U.S. patent application Ser. No. 16/020,115, filed Jun. 27, 2018, U.S. Publication No. 2018/0368823, the content of which is incorporated herein by reference in its entirety.

This application is related to U.S. Provisional Application No. 61/818,878, filed May 2, 2013, the content of which is incorporated herein by reference in its entirety.

This application is related to PCT Application No. PCT/US2014/036571, filed May 2, 2014, PCT Publication No. WO2014/179683, the content of which is incorporated herein by reference in its entirety.

This application is related to U.S. patent application Ser. No. 14/888,189, filed Oct. 30, 2015, U.S. Publication No. 2016/0067000, now U.S. Pat. No. 9,913,695, issued on Mar. 13, 2018, the content of which is incorporated herein by reference in its entirety.

This application is related to U.S. patent application Ser. No. 15/916,664, filed Mar. 9, 2018, U.S. Publication No. 2018/0256269, the content of which is incorporated herein by reference in its entirety.

This application is related to U.S. Provisional Application No. 61/909,605, filed Nov. 27, 2013, the content of which is incorporated herein by reference in its entirety.

This application is related to U.S. Provisional Application No. 62/052,736, filed Sep. 19, 2014, the content of which is incorporated herein by reference in its entirety.

This application is related to PCT Application No. PCT/US2014/067091, filed Nov. 24, 2014, PCT Publication No. WO2015/081008, the content of which is incorporated herein by reference in its entirety.

This application is related to U.S. patent application Ser. No. 15/038,531, filed May 23, 2016, U.S. Publication No. 2016/0287224, the content of which is incorporated herein by reference in its entirety.

This application is related to U.S. Provisional Application No. 62/008,453 filed Jun. 5, 2014, the content of which is incorporated herein by reference in its entirety.

This application is related to PCT Application No. PCT/US2015/034424, filed Jun. 5, 2015, PCT Publication No. WO2015/188071, the content of which is incorporated herein by reference in its entirety.

This application is related to U.S. patent application Ser. No. 15/315,868, filed Dec. 2, 2016, U.S. Publication No. 2017/0100197, the content of which is incorporated herein by reference in its entirety.

This application is related to U.S. patent application Ser. No. 16/225,156, filed Dec. 19, 2018, U.S. Publication No. 2019/xxxxxx, the content of which is incorporated herein by reference in its entirety.

This application is related to U.S. Provisional Application No. 62/150,223, filed Apr. 20, 2015, the content of which is incorporated herein by reference in its entirety.

This application is related to U.S. Provisional Application No. 62/299,249, filed Feb. 24, 2016, the content of which is incorporated herein by reference in its entirety.

This application is related to PCT Application No. PCT/US2016/028374, filed Apr. 20, 2016, PCT Publication No. WO2016/172162, the content of which is incorporated herein by reference in its entirety.

This application is related to U.S. patent application Ser. No. 15/567,109, filed Oct. 17, 2017, U.S. Publication No. 2018-0228557 the content of which is incorporated herein by reference in its entirety.

This application is related to U.S. Provisional Application No. 62/401,390, filed Sep. 29, 2016, the content of which is incorporated herein by reference in its entirety.

This application is related to PCT Application No. PCT/US2017/054297, filed Sep. 29, 2017, PCT Publication No. WO2018/064475, the content of which is incorporated herein by reference in its entirety.

This application is related to U.S. Provisional Application No. 62/517,433, filed Jun. 9, 2017, the content of which is incorporated herein by reference in its entirety.

This application is related to PCT Application No. PCT/US2018/036876, filed Jun. 11, 2018, PCT Publication No. WO2018/227180, the content of which is incorporated herein by reference in its entirety.

This application is related to U.S. Provisional Application No. 62/481,309, filed Apr. 4, 2017, the content of which is incorporated herein by reference in its entirety.

This application is related to U.S. Provisional Application No. 62/598,812, filed Dec. 14, 2017, the content of which is incorporated herein by reference in its entirety.

This application is related to U.S. Provisional Application No. 62/617,513, filed Jan. 15, 2018, the content of which is incorporated herein by reference in its entirety.

This application is related to PCT Application No. PCT/US2018/026016, filed Apr. 4, 2018, PCT Publication No. WO2018/187425 the content of which is incorporated herein by reference in its entirety.

This application is related to U.S. Provisional Application No. 62/533,644, filed Jul. 17, 2017, the content of which is incorporated herein by reference in its entirety.

This application is related to U.S. Provisional Application No. 62/614,263, filed Jan. 5, 2018, the content of which is incorporated herein by reference in its entirety.

This application is related to PCT Application No. PCT/US2018/042449, filed Jul. 17, 2018, PCT Publication No. WO2019/xxxxxx, the content of which is incorporated herein by reference in its entirety.

This application is related to U.S. Provisional Application No. 62/582,283, filed Nov. 6, 2017, the content of which is incorporated herein by reference in its entirety.

This application is related to U.S. Provisional Application No. 62/614,346, filed Jan. 5, 2018, the content of which is incorporated herein by reference in its entirety.

This application is related to PCT Application No. PCT/US2018/059338, filed Nov. 6, 2018, PCT Publication No. WO2019/xxxxxx, the content of which is incorporated herein by reference in its entirety.

This application is related to U.S. Design Application No. 29/632,148, filed Jan. 5, 2018, the content of which is incorporated herein by reference in its entirety.

This application is related to U.S. Pat. No. 9,011,318, issued Apr. 21, 2015, the content of which is incorporated herein by reference in its entirety.

### FIELD

The present inventive concepts generally relate to the field of medical robotic systems, and more particular, to a surgeon console from where an operator can perform robotic-assisted medical procedures.

### BACKGROUND

As less invasive medical techniques and procedures become more widespread, medical professionals such as surgeons may require articulating surgical tools, such as endoscopes, to perform such less invasive medical techniques and procedures that require access to locations within the patient, such as a site accessible through the mouth or other natural orifice, or a site accessible through an incision through the patient's skin.

There is a need for improved systems for performing a medical procedure.

### SUMMARY

In an aspect, a system for performing a medical procedure on a patient, comprises: an articulating probe assembly, comprising: an inner probe comprising multiple articulating inner links; an outer probe surrounding the inner probe and comprising multiple articulating outer links; and at least two working channels that exit a distal portion of the probe assembly; at least one tool configured to translate through one of the at least two working channels; and a user interface for controlling the articulating probe assembly.

In an embodiment, the user interface comprises at least one controller for controlling the articulating probe assembly.

In an embodiment, the user interface comprises a hands-free mechanism for moving the at least one controller out of operating position to gain access to the patient.

In an embodiment, the dimensions of the user interface are constructed and arranged to allow the surgeon direct access to the patient once the controllers are moved out of the operating position.

In an embodiment, a height of the controllers is constructed and arranged to accommodate a seated operating position.

In an embodiment, a height of the controllers is constructed and arranged to accommodate a standing operating position.

In an embodiment, the at least one controller is constructed and arranged to sense the presence of a hand on the at least one controller.

In an embodiment, the at least one controller comprises a pitch angle that is constructed and arranged to maximize a line of sight for a surgeon.

In an embodiment, a height of the at least one controller is constructed and arranged to be adjusted.

In an embodiment, the user interface comprises at least one foot pedal.

In an embodiment, the at least one foot pedal is detachable.

### BRIEF DESCRIPTION OF THE DRAWINGS

The foregoing and other objects, features and advantages of embodiments of the present inventive concepts will be apparent from the more particular description of preferred embodiments, as illustrated in the accompanying drawings in which like reference characters refer to the same elements throughout the different views. The drawings are not necessarily to scale, emphasis instead being placed upon illustrating the principles of the preferred embodiments.

FIG. 1 is a schematic view of a system in which embodiments of the present inventive concepts can be practiced.

FIGS. 1A-C are graphic demonstrations of a robotic probe, in accordance with embodiments of the present inventive concepts.

FIG. 2 is a perspective view of a surgeon console, in accordance with embodiments of the present inventive concepts.

FIG. 3 is a rear view of the surgeon console of FIG. 2, in accordance with embodiments of the present inventive concepts.

FIG. 4 is a top view of the surgeon console of FIGS. 2 and 3, in accordance with embodiments of the present inventive concepts.

FIG. 5A is a front view of the surgeon console of FIGS. 2-4 in a first position, in accordance with embodiments of the present inventive concepts.

FIG. 5B is a front view of the surgeon console of FIGS. 2-5A in a second position, in accordance with embodiments of the present inventive concepts.

FIG. 6 is a perspective view of an input device of FIGS. 2-5B, in accordance with embodiments of the present inventive concepts.

FIG. 6A is a perspective view of first and second input devices of a surgeon console, in accordance with embodiments of the present inventive concepts.

FIG. 7 is a perspective view of a surgeon console in an operating position, in accordance with embodiments of the present inventive concepts.

FIG. 8 is a perspective view of a surgeon console in a patient access position, in accordance with embodiments of the present inventive concepts.

FIG. 9 is a perspective view of a surgeon console in an operating position, in accordance with embodiments of the present inventive concepts.

### DETAILED DESCRIPTION OF EMBODIMENTS

Reference will now be made in detail to the present embodiments of the technology, examples of which are illustrated in the accompanying drawings. Similar reference numbers can be used to refer to similar components. However, the description is not intended to limit the present disclosure to particular embodiments, and it should be construed as including various modifications, equivalents, and/or alternatives of the embodiments described herein.

It will be understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third etc. can be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.).

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of one or more of these.

As used herein, the term “proximate” shall include locations relatively close to, on, in and/or within a referenced component, anatomical location, or other location.

Spatially relative terms, such as “beneath,” “below,” “lower,” “above,” “upper” and the like can be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence.

The term “and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

In this specification, unless explicitly stated otherwise, “and” can mean “or,” and “or” can mean “and.” For example, if a feature is described as having A, B, or C, the feature can have A, B, and C, or any combination of A, B, and C. Similarly, if a feature is described as having A, B, and C, the feature can have only one or two of A, B, or C.

The expression “configured (or set) to” used in the present disclosure can be used interchangeably with, for example, the expressions “suitable for”, “having the capacity to”, “designed to”, “adapted to”, “made to” and “capable of” according to a situation. The expression “configured (or set) to” does not mean only “specifically designed to” in hardware. Alternatively, in some situations, the expression “a device configured to” can mean that the device “can” operate together with another device or component.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross-sectional area as the cross section of the component being described.

The terms “major axis” and “minor axis” of a component where used herein are the length and diameter, respectively, of the smallest volume hypothetical cylinder which can completely surround the component.

It is appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable sub-combination. For example, it will be appreciated that all features set out in any of the claims (whether independent or dependent) can be combined in any given way.

It is to be understood that at least some of the figures and descriptions of the invention have been simplified to focus on elements that are relevant for a clear understanding of the invention, while eliminating, for purposes of clarity, other elements that those of ordinary skill in the art will appreciate can also comprise a portion of the invention. However, because such elements are well known in the art, and because they do not necessarily facilitate a better understanding of the invention, a description of such elements is not provided herein.

Terms defined in the present disclosure are only used for describing specific embodiments of the present disclosure and are not intended to limit the scope of the present disclosure. Terms provided in singular forms are intended to include plural forms as well, unless the context clearly indicates otherwise. All of the terms used herein, including technical or scientific terms, have the same meanings as those generally understood by an ordinary person skilled in the related art, unless otherwise defined herein. Terms defined in a generally used dictionary should be interpreted as having meanings that are the same as or similar to the contextual meanings of the relevant technology and should not be interpreted as having ideal or exaggerated meanings, unless expressly so defined herein. In some cases, terms defined in the present disclosure should not be interpreted to exclude the embodiments of the present disclosure.

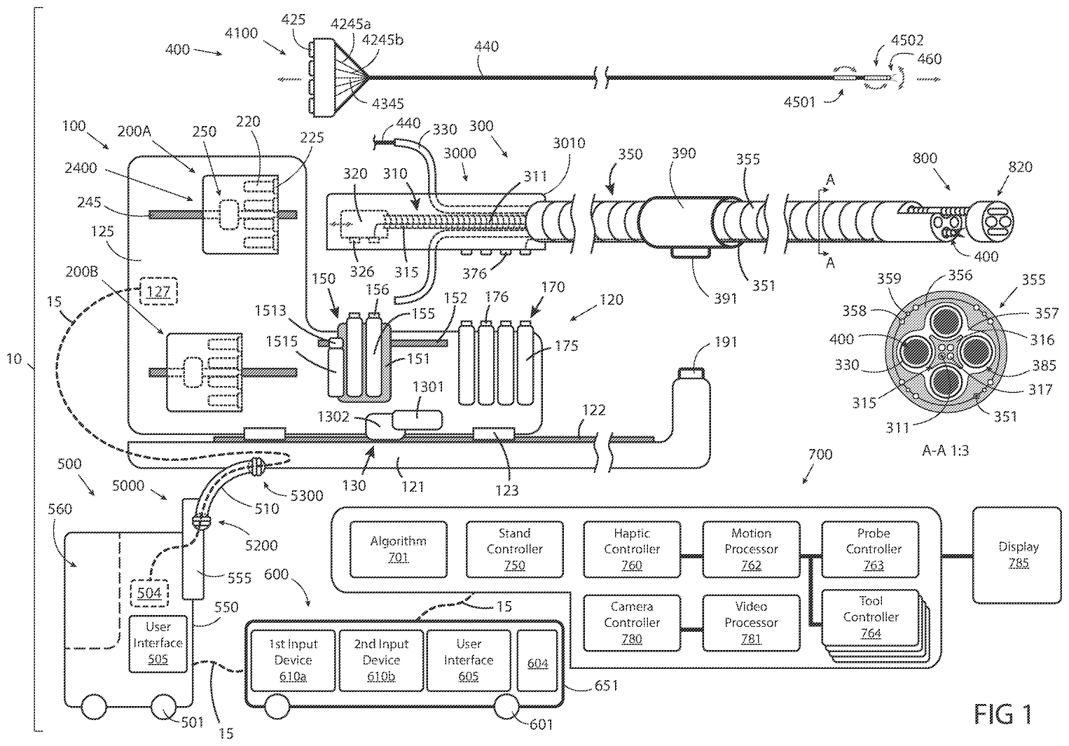

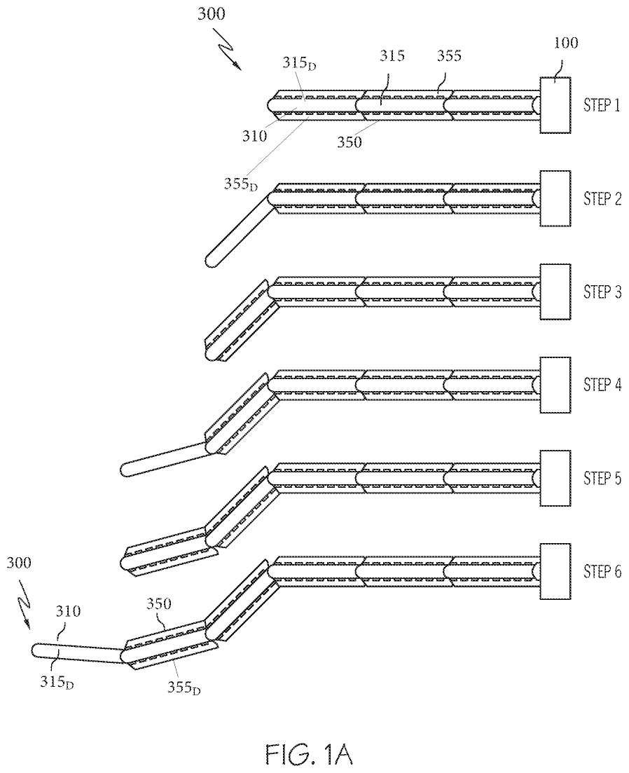

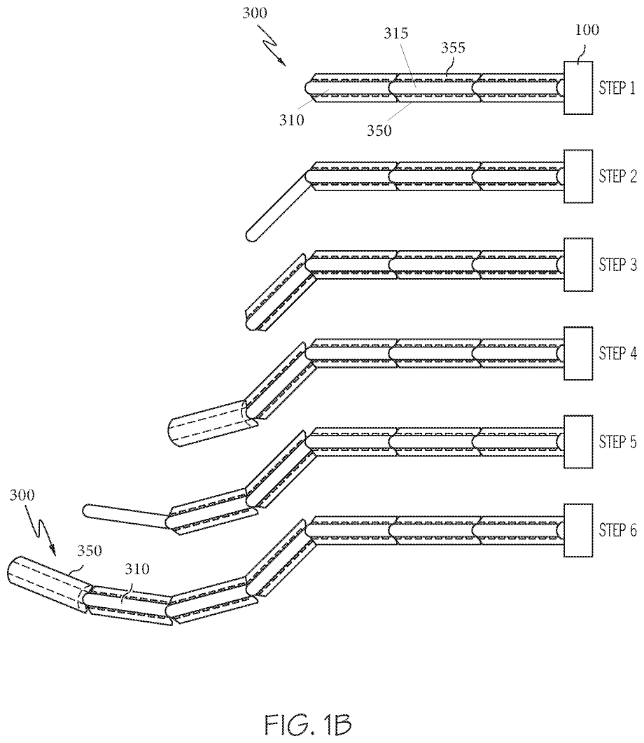

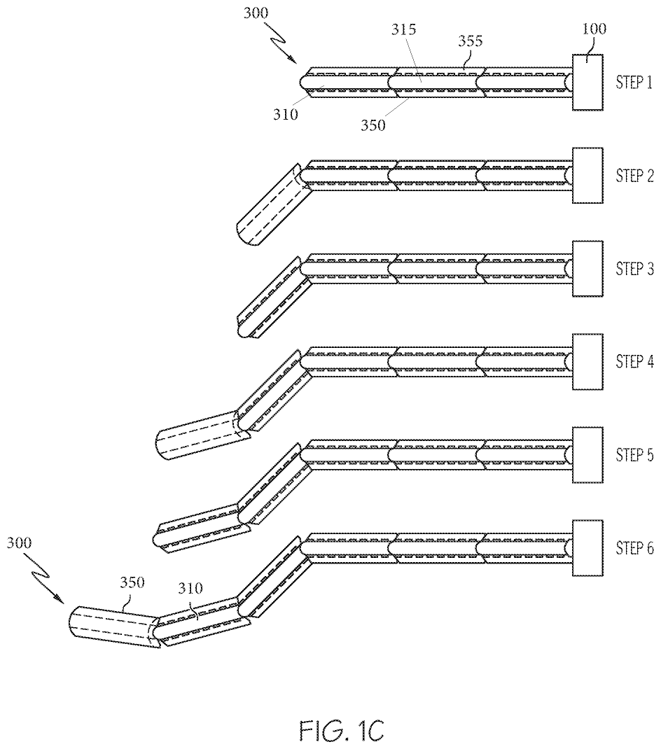

Referring to FIG. 1, a schematic view of a system in which embodiments of the present inventive concepts can be practiced is illustrated.

System 10 includes a robotic feeder 100. Feeder 100 interchangeably and operably engages a robotic probe assembly 300, and at least one robotic tool assembly 400. Feeder 100 is constructed and arranged to advance, retract, steer, and/or otherwise control the position and/or articulation of probe assembly 300 and/or tools 400, as described herein. One or more tools 400 can be slidingly received within a channel of probe assembly 300, and each tool 400 can be advanced beyond the distal end of probe assembly 300. Feeder 100 includes a probe manipulation assembly 120 for operably controlling the position and articulation of probe assembly 300. Feeder 100 also includes at least one tool manipulation assembly, tool drive 200 (e.g. tool drives 200A and 200B shown), for controlling the position and articulation of an attached tool 400. System 10 further includes a multi-dimensional positioning assembly, robotic stand 500. Stand 500 includes an articulation assembly 5000 for positioning feeder 100 with multiple degrees of freedom, for example within an operating room, relative to a patient and/or patient bed, as described herein. System 10 further includes a control interface, surgeon console 600, configured to receive commands from one or more operators of system 10 (e.g. one or more surgeons or other clinicians). Console 600 can include a first and second input device, 610A and 610B respectively (singly or collectively input devices 610 herein), each configured to receive multi-dimensional input data (e.g. via a kinematic input device as described herein). System 10 further includes a collection of data processing components, collectively processing unit 700. Processing unit 700 can include one or more algorithms, controllers, memory, state machines, and/or processors, singly and/or collectively controlling one or more components of system 10 (e.g. based at least on one or more user inputs received by one or more input components of system 10). System 10 further includes an imaging device, camera assembly 800 (e.g. a tool 400 configured as a camera, as described herein), comprising one or more cameras, camera 820. Image data (e.g. still and/or video images) captured by camera 820 can be displayed on one or more monitors or other screens, display 785. One or more components described herein as included in a tool 400 can also be included in camera assembly 800, for example camera assembly 800 can comprise a tool 400 with camera 820 operably attached thereto. A conduit, bus 15, can connect one or more components of system 10. Bus 15 can comprise one or more electrical, fluid, optical, and/or other conduits for transferring information, power, one or more fluids, light energy, and combinations of one or more of these.

### Probe Assembly 300

Probe assembly 300 includes an outer probe 350, comprising multiple articulating outer links 355. Links 355 each comprise a ring-like structure (e.g. a hollow tube-like structure), link body 356, surrounding a hollow bore, channel 357. Collectively, channels 357 define a lumen extending along at least a portion of the length of outer probe 350. Links 355 can include multiple lumens extending therethrough, such as lumens extending along the link, through link body 356. For example, links 355 can include one or more steering cable lumens, lumens 358, such as eight lumens 358 shown. Lumens 358 can each slidingly receive a steering cable 351 that is used to control at least the articulation of outer probe 350, as described herein. Links 355 can also include one or more auxiliary lumens, four lumens 359 shown. In some embodiments, lumens 359 can slidingly receive elongate devices and/or filaments, such as optical fibers for delivering light to a surgical site.

Probe assembly 300 further includes inner probe 310, comprising multiple articulating inner links 315. Inner probe 310 is slidingly received within channels 356 extending through outer probe 350. Links 315 can comprise a link body 316, and can include multiple lumens extending therethrough, such as lumens extending along the link. For example, links 315 can include one or more steering cable lumens, lumens 317, such as four lumens 318 shown. Lumens 317 can each slidingly receive a steering cable 311 used to control at least the articulation of inner probe 310, as described herein.

The outer shape of link body 316 can align with the shape of the channel 357 to form a plurality of passageways or working channels 385, extending throughout probe assembly 300. Working conduits 330 can be slidingly received within channels 385, extending throughout the probe assembly 300. Each conduit 330 can sliding receive at least a portion of a tool 400.

Probe assembly 300 can be of similar construction and arrangement to the similar device described in reference to applicant's co-pending U.S. patent application Ser. No. 16/114,681, filed Aug. 28, 2018, the content of which is incorporated herein by reference in its entirety.

Probe assembly 300 further comprises a manipulation assembly 3000, operably attached to the proximal portion of probes 310, 350. Manipulation assembly 3000 comprises a housing 3010, surrounding at least a cart 320, operably attached to inner probe 310. Manipulation assembly 3000 comprises one or more bobbins 376 operably attached to one or more steering cables 351 (also referred to herein as control cables). Cart 320 comprises one or more bobbins 326 operably attached to one or more steering cables 311. Manipulation assembly 3000 is constructed and arranged to operably and removably attach to feeder 100, as described herein. Manipulation assembly 3000 supports the proximal sections of one or more working conduits 330 in an orientation that is radially dispersed relative to the radially compact orientation of the distal portions of working conduits 330 within probe assembly 300.

Probe assembly 300 can include a support structure, introducer 390. Introducer 390 can comprise a rigid elongate structure. Introducer 390 can surround at least a portion of probe assembly 300. Introducer 390 can comprise a connector portion 391, constructed and arranged to operably attach to a portion of feeder 100 as described herebelow. Probe assembly 300 can be of similar construction and arrangement to the similar device described in applicant's co-pending application U.S. Provisional Application No. 62/614,240, filed Jan. 5, 2018, the content of which is incorporated herein by reference in its entirety.

### Feeder 100

Feeder 100 comprises a manipulation assembly 120 comprising a carriage 125 operably attached to a base 121. Carriage 125 can comprise one or more linear bearings 123 fixedly attached thereto, slidingly attached to a linear rail assembly 122, which in turn is fixedly attached to base 121. Linear rail assembly 122 can comprise one or more rails and/or lead screws. Manipulation assembly 120 can comprise a linear drive assembly 130, that is operably attached to carriage 125 and linear rail assembly 122. For example, linear rail assembly 122 can comprise at least a lead screw, and linear drive assembly 130 can comprise a motor 1301 and gear box 1302. Linear drive assembly 130 can be configured to engage the lead screw of linear rail assembly 122, such as to translate carriage 125 relative to base 121.

Manipulation assembly 120 can comprise a probe support assembly 170. Probe support assembly 170 can comprise at least a portion of carriage 125. Probe support assembly 170 can comprise one or more motors 175, each operably attached to a capstan 176. Probe support assembly 170 is constructed and arranged to operably and removably attach to manipulation assembly 3000, for example, such that each capstan 176 operably engages a corresponding bobbin 376. Motors 175 can be configured to rotate capstans 176, which in turn rotate bobbins 376, tensioning and de-tensioning cables 351 to control the articulation of outer probe 350.

Probe support assembly 170 can further comprise a probe translation assembly 150. Probe translation assembly 150 can comprise one or more motors 155, each operably attached to a capstan 156. Probe translation assembly 150 is constructed and arranged to operably and removably attach to cart 320, for example such that each capstan 156 operably engages a corresponding bobbin 326. Motors 155 can be configured to rotate capstans 156, which in turn rotate bobbins 326, tensioning and de-tensioning cables 311 to control the articulation of inner probe 310. Probe translation assembly 150 can comprise a cart 151. Motors 155 can be fixedly attached to cart 151. Cart 151 can be slidingly attached to a linear rail assembly 152, fixedly attached to carriage 125. Linear rail assembly 152 can comprise one or more rails and/or lead screws. Probe translation assembly 150 can comprise a motor 1515 and drive gear 1513 operably attached thereto. Drive gear 1513 can operably attach to linear rail assembly 152, for example when linear rail assembly 152 comprises at least a lead screw. Motor 1515 can be configured to rotate drive gear 1513 to translate cart 151 relative to carriage 125. Cart 151 can be constructed and arranged to engage cart 320, such that translation of cart 151 causes the translation of cart 320 within manipulation assembly 3000. Translation of cart 320 can cause the translation of inner probe 310 with respect to outer probe 350, as described herein.

Feeder 100 can include a connector portion 191, constructed and arranged to removably connect to introducer 390 of probe assembly 300. Connector portion 191 can be positioned at the distal end of carriage 125, as shown.

Feeder 100 can include one or more modules 127, such as one or more processors and/or controllers. Module 127 can be operably attached to one or more components of system 10 via bus 15.

Feeder 100 can be of similar construction and arrangement to the similar device described in applicant's co-pending application U.S. Provisional Application No. 62/614,240, filed Jan. 5, 2018, the content of which is incorporated herein by reference in its entirety.

### Tool Drive 200

Each tool drive 200 (also referred to herein as a singular tool drive 200) is configured to operably and interchangeably attach to one or more tools 400. Feeder 100 can comprise one, two, three, four, or more tool drives, tool drives 200A and 200B shown. Additional tool drives can be mounted to carriage 125 opposite tool drives 200A and 200B (e.g. on the opposite side of carriage 125). Tool drive 200 can slidingly attach to carriage 125 via a translation assembly 2400. Translation assembly 2400 can comprise a linear rail assembly 245, fixedly attached to carriage 125. Linear rail assembly 245 can comprise one or more rails and/or lead screws. Translation assembly 2400 can further comprise a linear drive assembly 250, operably attached to tool drive 200 and linear rail assembly 245. For example, linear rail assembly 245 can comprise at least a lead screw, and linear drive assembly 250 can comprise a motor and/or a gear box. Linear drive assembly 250 can be configured to engage the lead screw of linear rail assembly 245, to translate tool drive 200 relative to carriage 125. Translation of tool drive 200 can cause the translation of an attached tool 400, for example relative to outer probe 350 operably attached to manipulation assembly 120.

Tool drive 200 can comprise one or more motors 220, configured to manipulate one or more components of tool drive 200. For example, one or more motors 220 can be configured to rotate one or more assemblies of tool drive 200 relative to each other, and/or to rotate one or more gears 225 (e.g. capstans) of tool drive 200. Gears 225 of tool drive 200 can be configured to operably engage one or more bobbins of an attached tool 400, as described herein, to control the articulation of the attached tool 400.

Tool drive 200 can be of similar construction and arrangement to the similar device described in applicant's co-pending application U.S. Provisional Application No. 62/614,228, filed Jan. 5, 2018, the content of which is incorporated herein by reference in its entirety.

### Tool 400

Tool 400 can include a manipulation assembly 4100, operably attached to the proximal end of a shaft 440. Shaft 440 can comprise a flexible shaft, comprising one or more lumens. Tool 400 can comprise one or more sets of steering (or control) cables 4245a, 4245b, and or 4345. Cables 4245a,b can be operably attached to manipulation assembly 4100, and extend through shaft 440 to a first and second articulation section 4501 and 4502, respectively. Cables 4245a,b can be tensioned and/or de-tensioned by manipulation assembly 4100 to cause the articulation of articulation sections 4501 and 4502, respectively. Cables 4345 can be operably attached to manipulation assembly 4100, and extend through shaft 440 to an end effector 460. Cables 4345 can be tensioned and/or de-tensioned by manipulation assembly 4100 to cause the articulation or other manipulation of end effector 460. System 10 can comprise multiple tools 400, such as four, five, six, or more tools 400, each exchangeable and operably attachable to tool drives 200. End effectors 460 can comprise scissors, graspers, blades, cautery devices, laser devices, and the like. Manipulation assembly 4100 can be constructed and arranged to removably attach to tool drive 200, such that gears 225 engage bobbins 425 of manipulation assembly 4100. Motors 220 of tool drive 200 can rotate gears 225, and bobbins 425, to tension and/or de-tension one or more cables of tool 400 described herein, to tension and/or de-tension the cables and manipulate tool 400. Manipulation assembly 4100 can also be constructed and arranged to rotate one or more components of tool 400 relative to each other, for example to rotate end effector 460 relative to shaft 440.

Tool 400 can be of similar construction and arrangement to the similar device described in applicant's co-pending application U.S. Provisional Application No. 62/614,225, filed Jan. 5, 2018, the content of which is incorporated herein by reference in its entirety.

### Camera Assembly 800

In some embodiments, as described hereabove, a tool 400 can be configured as a camera assembly 800. Camera assembly 800 can comprise a camera 820, operably attached to the distal end of shaft 440 of a tool 400. In some embodiments, camera 820 is attached to shaft 440 after shaft 440 has been inserted through probe assembly 300. For example, in some embodiments, camera 820 is larger than working channel 385.

Camera assembly can be of similar construction and arrangement to the similar device described in applicant's co-pending PCT International Patent Application No. PCT/US2018/059338, filed Nov. 6, 2018, the content of which is incorporated herein by reference in its entirety.

### Stand 500

Stand 500 can be constructed and arranged to position feeder 100 relative to a patient and/or patient bed, such as to position probe assembly 300 for a surgical procedure. For example, surgical procedures can include but are not limited to transabdominal procedures, transoral procedures, trans anal procedures, and/or trans vaginal procedures. Stand 500 includes a base 550, supporting an articulation assembly 5000. Articulation assembly 5000 includes a tower 555, extending vertically from base 550. A first hub 5200 is operably attached to tower 555. First hub 5200 can be adjusted along the height of tower 555, via one or more motors and/or vertical translation assemblies. First hub 5200 is operably attached to positioning arm 510, which is operably attached to a second hub 5300. Second hub 5300 is operably attached to base 121 of feeder 100. Hubs 5200 and 5300 can each comprise one or more motors, gears, hinges, axles, and the like, configured to manipulate the position of feeder 100 relative to stand 500. Bus 15 of system 10 can operably connect feeder 100 to stand 500. In some embodiments, bus 15 is routed through hubs 5200, 5300, arm 510, and/or tower 555, such that bus 15 is at least partially contained within articulation assembly 5000.

Stand 500 can comprise a recess 560. Articulation assembly 5000 can be configured to “fold” into a stowed position, with feeder 100 positioned at least partially within recess 560. In some embodiments stand 500 can comprise a processor 504 and a user interface 505. User interface 505 can include input and output functionality, such as a touchscreen monitor. User interface 505 can be configured to allow a user to control one or more components of system 10, for example the articulation of articulation assembly 5000. In some embodiments, stand 500 includes one or more wheels 501, and is constructed and arranged to be mobile. For example, stand 500 can be manually repositionable by a user and/or can be robotically repositionable, for example when wheels 501 are driven by one or more motors.

Stand 500 can be of similar construction and arrangement to the similar device described in applicant's co-pending application U.S. Provisional Application No. 62/614,223, filed Jan. 5, 2018, the content of which is incorporated herein by reference in its entirety.

### Surgeon Console 600

Surgeon console 600 can be operably attached to one or more components of system 10, such as via bus 15. Console 600 can comprise a base 651, supporting input devices 610a,b, and user interface 605. Console 600 can comprise a processor 604. In some embodiments, processor 604 can receive commands from input device 610a,b, and/or user interface 605. User interface 605 can be configured to allow a user to control one or more components of system 10. In some embodiments, user interface 605 can be a redundant interface of user interface 505, such that a user can perform the same operations from either interface. In some embodiments, console 600 includes one or more wheels 601, and is constructed and arranged to be mobile. For example, console 600 can be manually repositionable by a user and/or can be robotically repositionable, for example when wheels 601 are driven by one or more motors.

Console 600 can be of similar construction and arrangement to the similar device described in applicant's co-pending application U.S. Provisional Application No. 62/614,224, filed Jan. 5, 2018, the content of which is incorporated herein by reference in its entirety.

### Processor 700

Processing unit 700 can comprise one or more controllers and/or processors, located throughout system 10. For example, processor 700 can comprise a computer or other processing device, and/or can comprise one or more controllers or modules of system 10 (e.g. module 127 of feeder 100, processor 504 of stand 500, and/or processor 604 of user interface 600). Processing unit 700 can comprise one or more algorithms for processing data and/or commanding one or more components of system 10 to perform one or more operations. Processing unit 700 can comprise one or more controllers for controlling components of system 10. Processing unit 700 can comprise a stand controller 750, for operational control of stand 500. Processing unit 700 can comprise a camera controller, for operational control of camera assembly 800. Camera controller 780 can be operably attached to a video processor 781 for processing image data captured by camera 820. Video processor 781 can provide processed image data to a display 785, for display to a user. Processing unit 700 can comprise a haptic controller 760, operably attached to input devices 610a,b of console 600, for example via processor 604. Haptic controller 760 can be operably attached to a motion processor 762, which is operably attached to a probe controller 763, and one or more tool controllers 764. Haptic controller 760 can receive multi-dimensional input data (e.g. via a kinematic input device) from input devices 610a,b, and/or provide haptic feedback commands to input devices 610a,b. Motion processor 762 can process the multi-dimensional input data, and provide articulation and/or translation commands to probe controller 763 and/or tool controllers 764. Probe controller 763 can provide commands to one or more motors of system 10, for example to one or more motors of manipulation assembly 120 to at least advance, retract, steer, and/or otherwise control the position and/or articulation of probe assembly 300. Tool controllers 764 can provide commands to one or more motors of system 10, for example one or more motors of a tool drive 200 to at least advance, retract, steer, and/or otherwise control the position and/or articulation of an attached tool 400.

Processor 700 can be of similar construction and arrangement to the similar device described in applicant's co-pending application U.S. Provisional Application No. 62/614,235, filed Jan. 5, 2018, the content of which is incorporated herein by reference in its entirety.

Referring additionally to FIGS. 1A-C, graphic demonstrations of a robotic probe 300 are illustrated, consistent with the present inventive concepts. Articulating probe 300 comprises essentially two concentric mechanisms, an outer mechanism and an inner mechanism, each of which can be viewed as a steerable mechanism. Each of the components of probe 300 can comprise one or more sealing elements, such as to support an insufflation procedure. FIGS. 1A-C show the concept of how different embodiments of robotic probe 300 operate. Referring to FIG. 1A, the inner mechanism can be referred to as a first mechanism or inner probe 310. The outer mechanism can be referred to as a second mechanism or outer probe 350. Each mechanism can alternate between rigid and limp states. In the rigid mode or state, the mechanism is just that—rigid. In the limp mode or state, the mechanism is highly flexible and thus either assumes the shape of its surroundings or can be re-shaped. It should be noted that the term “limp” as used herein does not necessarily denote a structure that passively assumes a particular configuration dependent upon gravity and the shape of its environment; rather, the “limp” structures described in this application are capable of assuming positions and configurations that are desired by the operator of the device, and therefore are articulated and controlled rather than flaccid and passive.

In some embodiments, one mechanism starts limp and the other starts rigid. For the sake of explanation, assume outer probe 350 is rigid and inner probe 310 is limp, as seen in step 1 in FIG. 1A. Now, inner probe 310 is both pushed forward by feeder 100, and a distal-most inner link 315D is steered, as seen in step 2 in FIG. 1A. Now, inner probe 310 is made rigid and outer probe 350 is made limp. Outer probe 350 is then pushed forward until a distal-most outer link 355D catches up to the distal-most inner link 315D (e.g. outer probe 350 is coextensive with inner probe 310), as seen in step 3 in FIG. 1A. Now, outer probe 350 is made rigid, inner probe 310 limp, and the procedure then repeats. One variation of this approach is to have outer probe 350 be steerable as well. The operation of such a device is illustrated in FIG. 1B. In FIG. 1B it is seen that each mechanism is capable of catching up to the other and then advancing one link beyond. According to one embodiment, outer probe 350 is steerable and inner probe 310 is not. The operation of such a device is shown in FIG. 1C.

In medical applications, operation, procedures, and so on, once robotic probe 300 arrives at a desired location, the operator, such as a surgeon, can slide one or more tools through one or more working channels of outer probe 350, inner probe 310, or one or more working channels formed between outer probe 350 and inner probe 310, such as to perform various diagnostic and/or therapeutic procedures. In some embodiments, the channel is referred to as a working channel that can, for example, extend between first recesses formed in a system of outer links and second recesses formed in a system of inner links. Working channels may be included on the periphery of robotic probe 300, such as working channels comprising one or more radial projections extending from outer probe 350, these projections including one or more holes sized to slidingly receive one or more tools. As described with reference to other embodiments, working channels may be positioned on other locations extending from, on, in, and/or within robotic probe 300.

Inner probe 310 and/or outer probe 350 are steerable and inner probe 310 and outer probe 350 can each be made both rigid and limp, allowing robotic probe 300 to drive anywhere in three-dimensions while being self-supporting. Articulating probe 300 can “remember” each of its previous configurations and for this reason, robotic probe 300 can retract from and/or retrace to anywhere in a three-dimensional volume such as the intracavity spaces in the body of a patient such as a human patient.

Inner probe 310 and outer probe 350 each include a series of links, i.e. inner links 315 and outer links 355 respectively, that articulate relative to each other. In some embodiments, outer links 355 are used to steer and lock robotic probe 300, while inner links 315 are used to lock robotic probe 300. In a “follow the leader” fashion, while inner links 315 are locked, outer links 355 are advanced beyond the distal-most inner link 315D. Outer links 355 are steered into position by the system steering cables, and then locked by locking the steering cables. The cable of inner links 315 is then released and inner links 315 are advanced to follow outer links 355. The procedure progresses in this manner until a desired position and orientation are achieved. The combined inner links 315 and outer links 355 may include working channels for temporary or permanent insertion of tools at the surgery site. In some embodiments, the tools can advance with the links during positioning of robotic probe 300. In some embodiments, the tools can be inserted through the links following positioning of robotic probe 300.

One or more outer links 355 can be advanced beyond the distal-most inner link 315D prior to the initiation of an operator controlled steering maneuver, such that the quantity extending beyond the distal-most inner link 315D will collectively articulate based on steering commands. Multiple link steering can be used to reduce procedure time, such as when the specificity of single link steering is not required. In some embodiments, between 2 and 20 outer links can be selected for simultaneous steering, such as between 2 and 10 outer links or between 2 and 7 outer links. The number of links used to steer corresponds to achievable steering paths, with smaller numbers enabling more specificity of curvature of robotic probe 300. In some embodiments, an operator can select the number of links used for steering (e.g. to select between 1 and 10 links to be advanced prior to each steering maneuver).

In some embodiments, the surgeon console 600 comprises a splay function: a hands-free method of moving the input devices 610 out of the operating position to gain access to the patient, without moving the surgeon console 600. Manual or automated re-latching of the input devices 610 to the surgeon console 600 can be provided.

In some embodiments, the height of the input devices 610 can be adjusted to accommodate seated or standing operating positions.

In some embodiments, the surgeon console 600 has a low “step over” height to allow the surgeon access directly to the patient once the input devices 610 are splayed.

In some embodiments, the surgeon console 600 comprises detachable/stow-able foot pedals 656 that allow for custom foot pedal positioning on the floor or raised from the floor (e.g. suspended from the surgeon console 600). This allows for different surgeon positions including sitting with feet on the floor, standing, or sitting on a raised chair. This also allows the foot pedals 656 to be placed around other auxiliary equipment, like cauterization pedals.

In some embodiments, the surgeon console 600 is constructed and arranged for customization of input devices 610 central access to allow comfortable, side by side use of multiple input devices 610.

In some embodiments, the addition of inputs/outputs on the input device 610 allows for activation (scope and instrument control) without removing a hand from input device 610. The user interface can sense the surgeon's hand on the input device 610.

In some embodiments, the pitch angle of the input devices 610 can be constructed and arranged to maximize and customize the user's visualization and line of sight.

In some embodiments, the height of the input devices 610 can be adjusted (e.g. to allow for personal preference and comfort). Individually adjustable input devices 610 can be provided for enhanced flexibility in positioning.

In some embodiments, the surgeon console 600 comprises an integrated cord management system for ease of transport and safety of personnel.

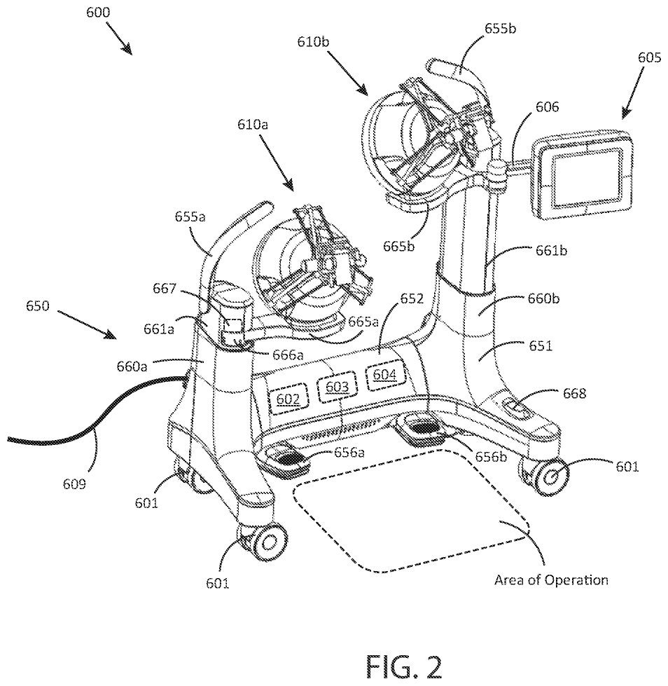

Referring to FIG. 2, a perspective view of a surgeon console 600 is illustrated, in accordance with embodiments of the present inventive concepts. The surgeon console 600 is constructed and arranged to control one or more robotic manipulator arms, probes, tools, and/or related devices on one or more different carts, for example, shown in FIG. 1.

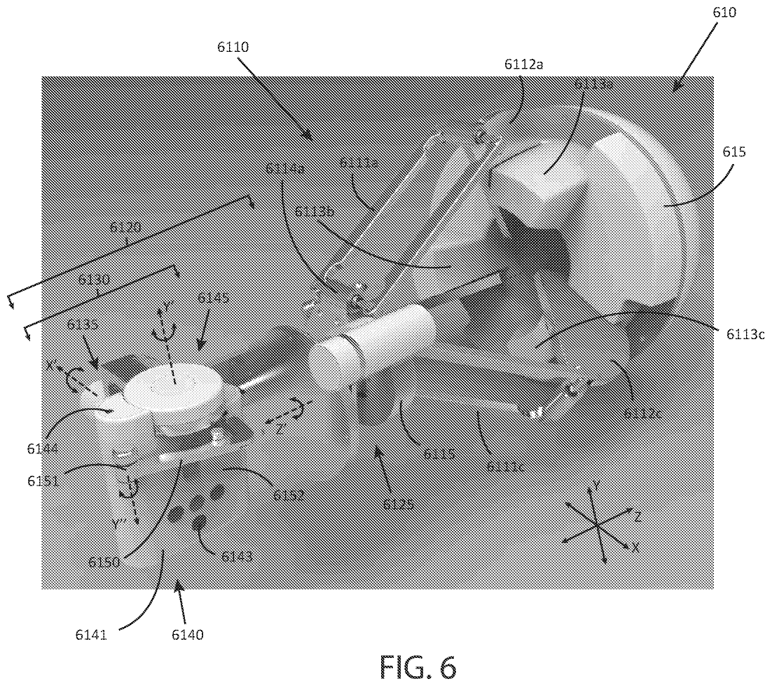

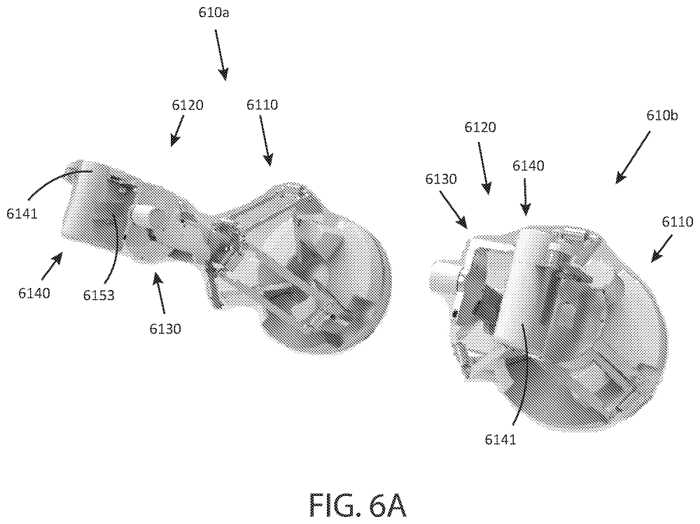

The surgeon console 600 comprises a stand 650, a first input device 610a, a second input device 610b each movably coupled to the stand 650, and a coupling for an auxiliary interface device, user interface 605. Each input device 610a, 610b (generally, 610) includes one or more controls, such as joysticks and/or buttons, for articulating various surgical tools and/or robotic probes to perform a medical procedure (e.g. a minimally-invasive or open surgery that is aided by the use of robotic systems on a patient). Input devices 610 are described in detail in reference to FIGS. 6 and 6A herebelow. Stand 600 can define an area of operation, where an operator sits and/or stands to operate input devices 610. In some embodiments, the surgery can be performed at a position that is opposite the area of operation. In FIG. 2, first input device 610a is manipulatable by a left hand of an operator, and second input device 610a is manipulatable by a right hand of the operator.

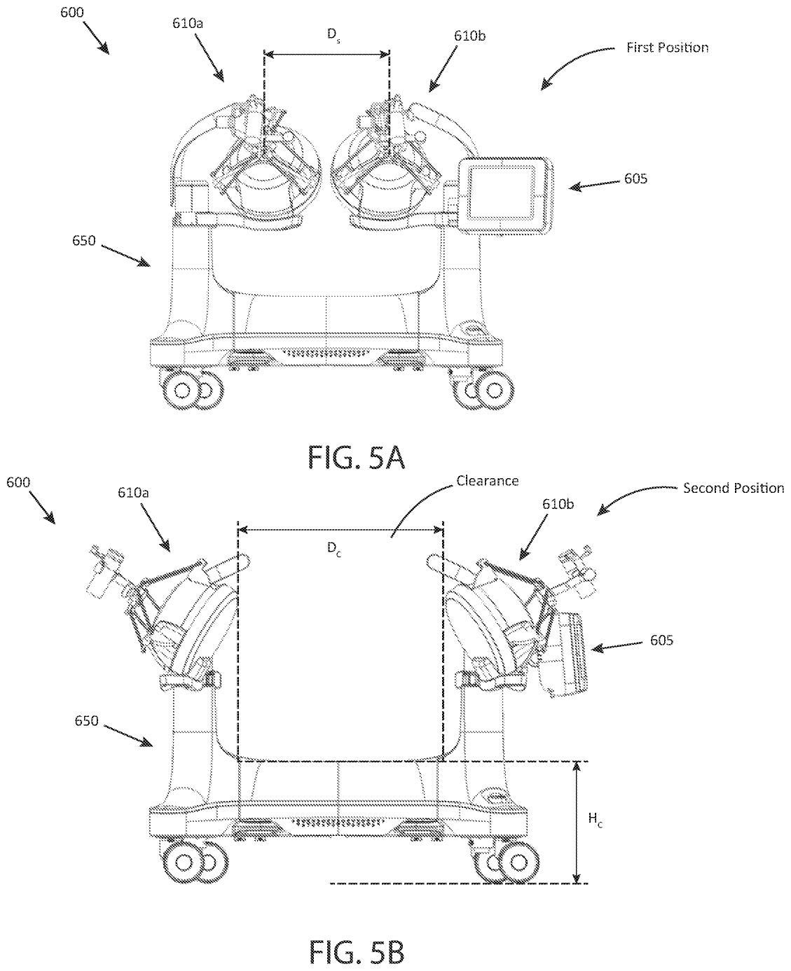

The surgeon console 600 is constructed and arranged to permit an operator to have quick accessibility to a patient in the event of an emergency or other situation where the operator must have direct physical contact with the patient. This accessibility is achieved by allowing for input devices 610a, 610b to be physically separated from each other (e.g. by any operator of system 10) to form an area of clearance between the input devices 610a, 610b (see FIG. 5B), allowing the operator of input devices 610a, 610b to gain direct access to the patient by passing through the area of clearance between the input devices 610a, 610b.

The stand 650 comprises a base 651 having a cross-member 652 extending between a first side of the base 651 and a second side of the base 651. A first arm 660a extends from a first side (e.g., the left side as shown in FIG. 2) of the base 651. A second arm 660b extends from a second, opposite side (e.g., the right side as shown in FIG. 2) of the base 651. In some embodiments, the cross-member 652 extends in a horizontal direction and the arms 660a, 660b (generally, 660) each extend vertically from the first and second sides, respectively.

The cross-member 652 can have a hollow interior (e.g. a hollow tubular configuration) and can extend between the first arm 660a and second arm 660b at a sufficiently low position so that the operator can easily step over the cross-member 652 during an emergency. The interior of the cross-member 652 can house one or more of a ballast 602, power supply 603, and a processor 604, and/or assorted cables, wires, electronic components, and/or other components that provide for operation of the console 600 and/or its input devices used for articulating various surgical tools and/or robotic probes.

The ballast 602 is configured to provide a sufficient mass and/or volume for providing the surgeon console 600 with a center of gravity that is located sufficiently low to provide an adequately stable platform of the console 600. Ballast 602 can be configured to resist undesirable movement of console 600 during use.

The power supply 603 is constructed and arranged to provide power to the console 600. Other power-generating components such as batteries, capacitors, and/or generators, can also be housed within the cross-member 652. In some embodiments, the console 600 does not comprise the power supply 603, such as when the console 600 receives power from an external source (e.g. an electrical outlet in a wall). The power supply 603 can provide a backup power source in the event of a power failure (e.g. power supply 603 stores energy for subsequent use).

The processor 604 can be configured to receive at least positional information from one or more controls of an input device 610, and transmit the positional information to a motion control processor (not shown) that is part of the console 600 or external to the console 600 but in electrical communication with the input devices 610.

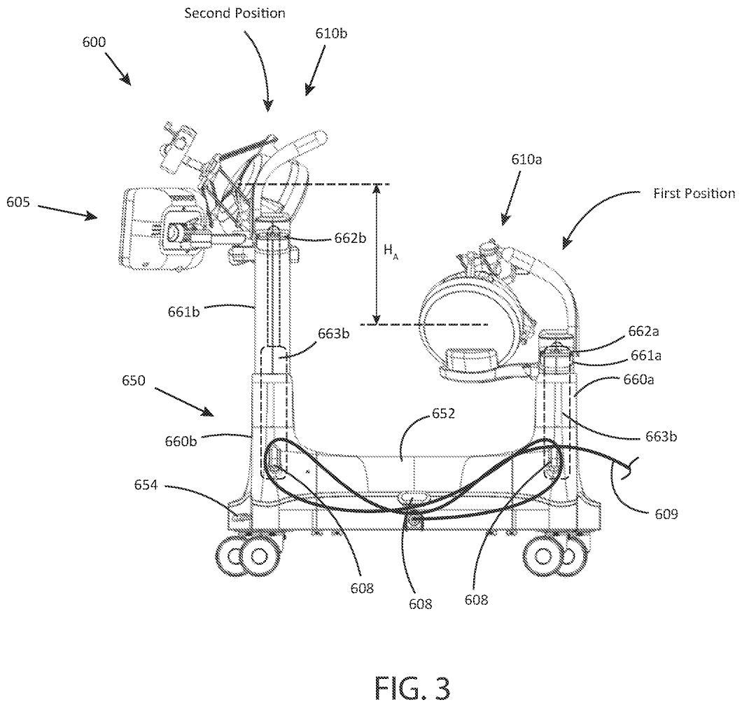

In some embodiments, a conduit 609 is in electrical communication with the processor 604, the input devices 610, power supply 602, and/or other electrical components for transmitting power, data, or a combination thereof. The console 600 can include one or more cable management elements 608 (as shown in FIG. 3) to allow the conduit 609 to be neatly organized, especially with respect to the cross-member 652, which is preferred to be clear of other components to reduce the risk of an operator tripping, stumbling, or otherwise prevented from quickly moving toward a patient in the event of an emergency when the input devices 610a, 610b are separated (see for example FIG. 5B). In some embodiments, cables such as the conduit 609 can extend from either side of the console 600.

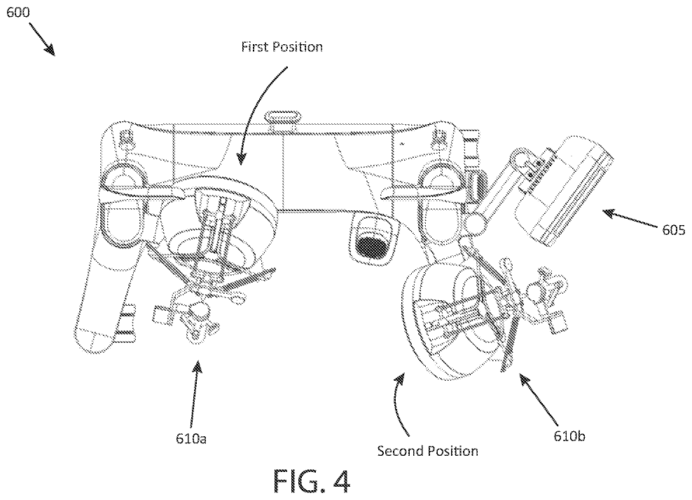

Referring to FIG. 3, a rear view of a surgeon console 600 is illustrated, in accordance with embodiments of the present inventive concepts. Referring additionally to FIG. 4, a top view of a surgeon console 600 is illustrated, in accordance with embodiments of the present inventive concepts. In some embodiments, each arm 660 includes a telescopic extension 661. For example, the telescopic extension 661 can include multiple extension tubes telescopically connected to each other to allow the extension 661 to expand and retract (e.g. expand and retract vertically). When expanded, the extension tubes are movably coupled so as to hold the telescopic extension 661 in place in the expanded position, and to support a weight applied to the extension 661 without causing collapse or inadvertent retraction of the extension tubes.

In some embodiments, arm 660a, 660b can include a spring 663a, 663b (generally, 663) such as a gas spring, piston, and/or other compressible element, for expanding and retracting the extension tubes of the extension 661 (e.g. providing a supporting force for retaining the extension 661 at a user-specific length, which includes full or partial expansion of the extension tubes). In some embodiments, an arm 660 can include a locking mechanism, control 662, for locking and unlocking the extension 661, for example, similar to a locking mechanism of a chair having a telescoping body that supports the weight of a user when in an expanded state.

Referring to FIGS. 5A and B, front views of a surgeon console 600 in a first position and a second position are illustrated, respectively, in accordance with embodiments of the present inventive concepts. Each extension 661a,b can include an articulating support arm 665a,b (generally, 665) that is rotatably attached by a hinge 666a,b (generally, 666). The support arms 665a,b are each constructed and arranged for supporting an input device 610a, 610b, respectively (generally, 610). The articulating support arms 665 can articulate between a first position, also referred to as an operating position, such as the operating position shown in FIG. 5A, and a second position, also referred to as an access position, such as the access position shown in FIG. 5B.

In some embodiments, one or more of the support arms 665a includes a biasing mechanism 667, such as a spring, proximate the hinge 666a that applies a force to the support arm 665a that biases the support arm 665a toward the second position (i.e., access position shown in FIG. 5B). In this manner, a force may be applied to move one or both input devices 610 from the second state to the first state (i.e., operator position). The support arm 665a can be locked into the first position (i.e., operating position) by a locking mechanism, which applies an opposite force to the biasing mechanism 667. Here, the support arm 665a can only be released by inactivating the locking mechanism, allowing the support arm 665a via the biasing mechanism 667 to transition from the first position to the second position. The locking mechanism can be inactivated, or released, by a release mechanism 668, such as a mechanical switch or the like, which communicates with the locking mechanism. The release mechanism 668 may be located at a foot of the base 651 and/or other location that is easily accessible by an operator or other person assisting the operator. An operator can use their foot, or hand, to engage release mechanism 668. The release mechanism 668 can include a computer processor that is configured to generate an electronic signal that is output to release the biasing mechanism 667 under predetermined conditions, thresholds, and/or user-defined requirements.

The input devices 610 can be height adjusted by way of the adjustable extensions 661, to accommodate for an operator, who can sit or stand, or have other height-related requirements. Also as shown in FIG. 3, the input devices 610a, 610b are independently adjustable, and can be adjusted a distance (H_{A}) due to the independent extension and retraction of the telescopic extensions 661a, 661b, respectively. Each of the input devices 610 can comprise a maximum height (e.g. when telescopic extensions 661a, 661b are fully extended) of approximately 43 inches, as measured to handpiece 6141 in a neutral position, and a minimum height (e.g. when telescopic extensions 661a, 661b are fully retracted) of approximately 32 inches, as measured to handpiece 6141 in a neutral position.

In some embodiments, the surgeon console 600 includes a set of wheels 601 coupled to the base 651 for allowing the surgeon console 600 to be transported to different locations (e.g. different operating rooms within a hospital and/or to a storage location). At least one wheel 601 can be pivotable (i.e., capable of rotating relative to the base 651). At least one wheel 601 can be locked by a caster wheel lock configuration or related braking mechanism. The locked wheel 601, in addition to the presence of the ballast 602, can prevent undesirable movement of the console 600 during use. In some embodiments, the console 600 includes at least one motor for moving the wheels 601. The motor can be in communication with a computer, which provides instructions (e.g. speed, direction, and the like) to the wheels 601 so that the stand 650 can be robotically positioned (e.g. the movement and/or relocation of stand 650 is not dependent on external forces provided by one or more operators).

In some embodiments, a handle 655a, 655b (generally, 655) is coupled to a telescopic extension 661a,b of a corresponding arm 660a, 660b. Each or both handles 655 can be used to position the console 600, relying on at least one pivotable wheel 601 to rotate the console 660, and/or to move the console 600 along a particular path of interest.

The arms 660 and cross-member 652 can define an area of operation, for example, shown in FIG. 2, where an operator can be positioned in front of the input devices 610. The console 600 can be positioned between the operator and a patient during a medical procedure performed by the operator. As shown in FIG. 5A, in the first position, the input devices 610a, 610b are positioned in front of the operator (i.e., between the operator and the patient, during a medical procedure). Here, a distance (Ds) between center regions of the first and second input devices 610a, 610b is established when in the first position during surgery. As shown in FIG. 5B, the arms 665a, 665b on which the input devices 610a, 610b are positioned, respectively, provide for an area of clearance between the area of operator and the patient, for example, a distance (Dc) between the input devices 610a, 610b shown in FIG. 5B, so that the operator can quickly move from the area of operation through the area of clearance to the patient. Dc can comprise a distance of at least 26.5 inches. The cross-member 652 extending between a first side of the base 651 and a second side of the base 651 has a sufficiently low clearance with respect to the ground so that the operator can step over the cross-member 652, for example, a height (H_{C}) shown in FIG. 5B. The cross-member 652 can comprise a height H_{C} of approximately 12 inches (e.g. the top surface of the cross-member 652 is approximately 12 inches from the ground).

In some embodiments, the stand 650 includes one or more foot-operated input devices, for example, two pedals 656a,b (generally, 656). A foot pedal 656 or related input device can communicate with, physically and/or electronically, with input devices present in an operating environment, which may or may not be part of the system 10. A foot pedal 656 can be detached from the stand 650, and interchanged with other foot pedals from an operating room or other location, such foot pedals used for fluoroscopy trigger and/or cautery trigger functions or the like, but not limited thereto. In some embodiments, a foot pedal 656 can be removed by a surgeon to position on the floor in the area of operation.

The console 600 is constructed and arranged to receive and hold in place an auxiliary interface device, user interface 605, such as a display that processes and displays a user interface or other visualizable information. The user interface 605 can include a touchscreen and/or standard display, and one or more peripheral or input/output devices such as a keyboard, mouse, indicator light, microphone, speaker, and so on. The user interface 605 can control one or more components of the system 10 shown in FIG. 1, such as the console 600, stand 500, probe 300, and/or other elements of the system 10. The user interface 605 can display one or more system parameters, such as an output from a video processor 781 (e.g., video images from a camera 820 of the system 10).

In some embodiments, the user interface 605 is positioned on a coupling apparatus, such as a bracket 606, which in turn is rotatably attached to the support arm 665, and can move (e.g., rotate) with the support arm 665. For example, when a support arm 665 is moved to a second, or access, position shown in FIG. 5B, the user interface 605 moves with the support arm 665. The bracket 606 can include mechanical components that interoperate to allow the bracket 606 to tilt, pan, and/or adjust to other positions of the user interface 605. The height of the user interface 605 can also be adjusted since the bracket 606 is coupled to the support arm 665, which in turn is part of a telescopic extension 661, which can be height-adjustable.

Referring to FIG. 6, a perspective view of an input device 610 of a surgeon console 600 is illustrated, in accordance with embodiments of the present inventive concepts. An input device 610 comprises a three-dimensional (3D) positioning assembly 6110, which includes a main base 615 and a first hub 6115. The main base 615 and a hub 6115 can be operably coupled by three sets of arms 6111a-c (generally, 6111), a set of blade hinges 6112a-c (generally, 6112), hinge assemblies 6113a-c, and a set of hinges 6114a-c (generally, 6114). For example, one blade hinge 6112 may be coupled to the main base 615 via hinge assembly 6113, and a hinge 6114 may be coupled to the hub 6115, and an arm 6111 may extend between the two hinges 6112 and 6114 to allow the hub 6115 to articulate relative to the main base 615.

The hub 6115 can be constructed and arranged to articulate relative to the main base 615 according to three degrees of freedom, which can include a movement of the hub 6115 along X, Y, and Z axes, in either direction of extension of any or all of the X, Y, and Z axes, or at angles therebetween.

In some embodiments, the input device 610 further comprises a first rotating assembly 6120 that is rotatably attached to the first hub 6115. In particular, first rotating assembly 6120 can rotate relative to first hub 6115 about a Z′ axis, for example, parallel to the Z axis. In some embodiments, the first rotating assembly 6120 rotates about a rotary joint assembly 6125.

In some embodiments, the input device 610 further comprises a second rotating assembly 6130 that is rotatably attached to a portion of the first rotating assembly 6120. In particular, second rotating assembly 6130 can rotate relative to the first rotating assembly 6120 about an X′ axis, for example, along or parallel to the X axis. In some embodiments, the second rotating assembly 6130 rotates about a rotary joint assembly 6135.

In some embodiments, the input device 610 further comprises a third rotating assembly 6140, for example, including a handle 6141, that is rotatably attached to a portion of the second rotating assembly 6130. In particular, third rotating assembly 6140 can rotate relative to the second rotating assembly 6130 about a Y′ axis, for example, parallel to the Y axis. In some embodiments, the second rotating assembly 6130 rotates about a rotary joint assembly 6145.

In some embodiments, the handle 6141 comprises a lever assembly 6150 that is rotatably attached to the handle 6141. The lever assembly 6150 can articulate relative to a main body of the handle 6141 about a hinge 6151, which extends along a Y″ axis parallel to the Y axis.

Each assembly 6125, 6135, 6145, 6151, and 6113 can include one or more rotary encoders, resistance and/or feedback motors, bearings, and/or other components known to those of ordinary skill in the art as being in the user input space to provide smooth motion along with haptic feedback to a user and provide a precise measurement of a user's movements.

In some embodiments, the lever assembly 6150 includes a trigger 6152 (e.g. a projection from 6150 for an operator to actuate 6150 with a finger) that, when depressed by an operator, rotates the lever assembly 6150 towards the handle 6141. In some embodiments, the trigger 6152 can comprise a strap or a ring like structure, that permits the operator to pull the trigger 6152 away from the handle 6141, (e.g. the operator can “close” and/or “open” lever 6150 relative to handle 6141) by pushing and/or pulling trigger 6152, respectively.

In some embodiments, as shown in FIG. 6A, the handle 6141 comprises an engagement portion 6153, on the opposite side of handle 6141 of the trigger 6152. During operation, an operator can grasp the handle 6141, engaging a thumb with the engagement portion 6153, and the forefinger with the trigger 6152. Engagement portion 6153 can include a similar strap and/or ring like structure for securing to the thumb. In these embodiments, a pinching and/or claw like motion (e.g. between the thumb and forefinger) actuates lever assembly 6150 with respect to handle 6141.

In some embodiments, the handle 6141 further comprises at least one control, button 6143, for example, toggle or instantaneous buttons or the like, on the trigger side, i.e., finger side and/or thumb side of the handle 6141. In some embodiments, the buttons 6143 can operate to alter the mapping of one or more DOFs of the input device 610, including switching tools being controlled from the input device 610.

In some embodiments, the handle 6141 includes one or more indicator lights 6144.

In a neutral position, or zero position, of the input device 610, for example, shown in FIGS. 6 and 6A, the first hub 6115 is positioned away from the base 615 so as to maximize a forward travel along the z direction, (e.g. where the first hub 6115 moves towards the base 615 due to a collapsing or expansion of the arms 6111-6113). Here, the possible travel in the backwards z direction (relative to the neutral position) is less than the possible forward travel.

Referring additionally to FIG. 6A, a detailed perspective view of a first and second input device 610 of a surgeon console is illustrated, in accordance with embodiments of the present inventive concepts. The two input devices 610a and 610b can be positioned relative to each other in approximately the first position described herein. As shown, the Z axis of each input device 610 can be angled slightly “downward”, such that an operator engages input devices 610 at a slightly downward angle, increasing visibility “over” input devices 610 (e.g. input devices 610 are not positioned directly in front of and/or in the direct line of sight of the operator during use). Input devices 610 are shown in the neutral position, handle 6141 positioned away from base 615 along the Z axis, with a relative “forward” motion being along the Z axis, such that input devices 610a,b can be positioned relatively close to each other (a distance Ds described hereabove in reference to FIG. 5A), without interference when articulated in the forward direction.

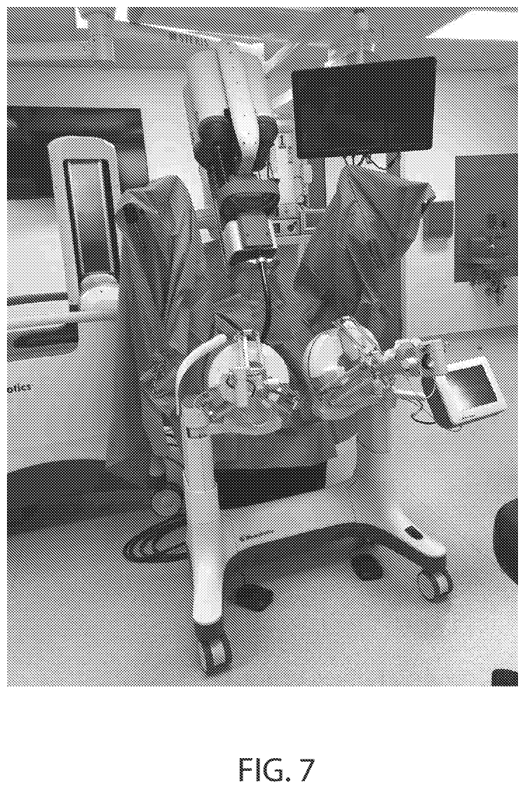

Referring additionally to FIG. 7, a perspective view of a surgeon console 600 in an operating position is illustrated, in accordance with embodiments of the present inventive concepts.

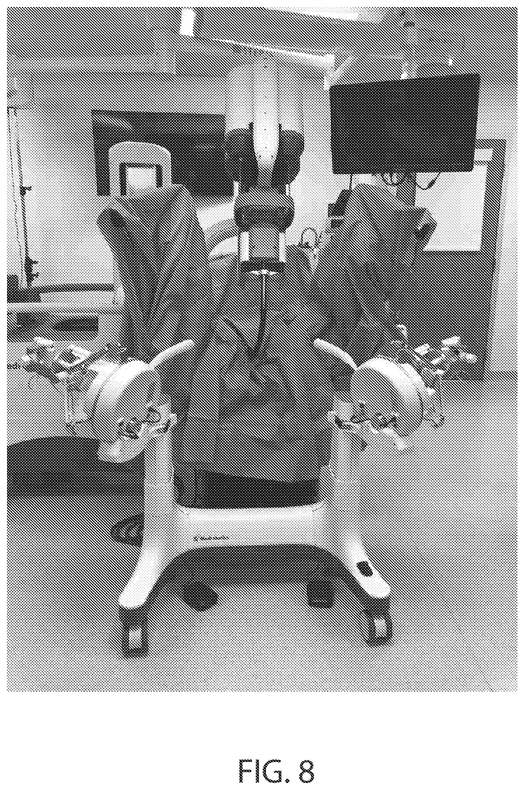

Referring additionally to FIG. 8, a perspective view of a surgeon console 600 in a patient access position is illustrated, in accordance with embodiments of the present inventive concepts.

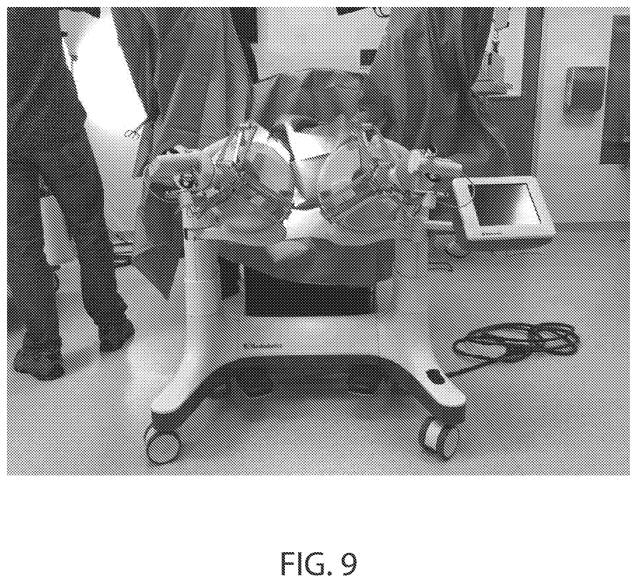

Referring additionally to FIG. 9, a perspective view of a surgeon console 600 in an operating position is illustrated, in accordance with embodiments of the present inventive concepts.

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

1. A system for performing a medical procedure on a patient, comprising:
an articulating probe assembly, comprising:
an inner probe comprising multiple articulating inner links;
an outer probe surrounding the inner probe and comprising multiple articulating outer links; and
at least two working channels that exit a distal portion of the probe assembly;
and
at least one tool configured to translate through one of the at least two working channels;
a user interface for controlling the articulating probe assembly.

2.-11. (canceled)
